(19) United States
(12) Patent Application Publication
(10) Pub. No.: US 2020/0120949 A1
(43) Pub. Date: Apr. 23, 2020

(54) FRUCTOOLIGOSACCHARIDE-CONTAINING OIL AND FAT, METHOD FOR PRODUCING THE SAME, FRUCTOOLIGOSACCHARIDE-CONTAINING OIL-BASED CONFECTIONERY, AND METHOD FOR PRODUCING THE SAME

(71) Applicant: MEIJI CO., LTD., Tokyo (JP)

(72) Inventors: Jerchyuan LIN, Tokyo (JP); Taku IMAI, Tokyo (JP); Daisuke HARADA, Tokyo (JP); Akiko ISHIDA, Tokyo (JP)

(73) Assignee: MEIJI CO., LTD., Tokyo (JP)

(21) Appl. No.: 16/658,449

(22) Filed: Oct. 21, 2019

(30) Foreign Application Priority Data
Oct. 23, 2018 (JP) 2018-199181

(51) Int. Cl. A23G 3/42; A23G 3/50; A23P 10/47; C07H 3/06

(52) U.S. Cl. CPC A23G 3/42; C07H 3/06; A23P 10/47; A23G 3/50

(57) ABSTRACT

It is an object of the present invention to provide a method and the like capable of producing a large amount of fructooligosaccharide-containing oil-based confectionery having low hygroscopicity. In the method for producing fructooligosaccharide-containing oil and fat according to the present invention, fructooligosaccharide is pulverized with at least the oil and fat and an emulsifier in a closed system pulverizer to obtain fructooligosaccharide-containing oil and fat. Then, a fructooligosaccharide-containing oil-based confectionery can be produced from the fructooligosaccharide-containing oil-based confectionery base obtained by mixing the fructooligosaccharide-containing oil and fat with the oil-based confectionery base or the raw material thereof. This fructooligosaccharide-containing oil-based confectionery has extremely low hygroscopicity, and if stored in a good environment, the moisture content can be kept extremely low at the point of 210 days from the time of production.

### PRIOR ART

The present invention relates to fructooligosaccharide-containing oil and fat and method for producing the same. Additionally, the present invention also relates to fructooligosaccharide -containing oil-based confectionery and method for producing the same. Furthermore, the present invention relates to oil-based confectionery, amorphous fructooligosaccharide -containing oil-based confectionery.

### BACKGROUND OF THE INVENTION

In the past, a fructooligosaccharide -containing chocolate has been proposed (see, for example, International Publication No. WO 2016/052721, Japanese Unexamined Patent Application Publication No. H07-252156, and the like).

### PRIOR-ART DOCUMENT

### Patent Document

[Patent Document 1] International Publication No. WO 2016/052721
[Patent Document 2] Japanese Unexamined Patent Application Publication No. H07-252156
[Patent Document 3] Japanese Unexamined Patent Application Publication No. H09 -65829

### SUMMERY OF THE INVENTION

### Problem to be Solved

Incidentally, when the above-mentioned fructooligosaccharide-containing chocolate is mass-produced by the usual method, the chocolate base increases in viscosity due to the high hygroscopicity caused by the fructooligosaccharide, and it becomes difficult to convey and form the chocolate base. Simply incorporating fructooligosaccharide into chocolate base does not satisfy the moisture value of 3% or less as stipulated in the Fair Competition Codes concerning Labeling on Chocolates (hereinafter referred to as the “chocolate code”).

Also, a chocolate produced using a crystal product of kestose, which is an ingredient of fructooligosaccharide (see, for example, Japanese Unexamined Patent Application Publication No. H09-65829, etc.) has excellent producing suitability and quality, but the sweetness thereof was lacking. In order to cope with various tastes of consumers, it is required to realize a balance of different sweetenesses.

It is an object of the present invention to provide a method and the like capable of producing a large amount of fructooligosaccharide-containing oil-based confectionery having low hygroscopicity. Another object of the present invention is to provide an oil-based confectionery having a good sweetening balance and low hygroscopicity.

### Means for Solving the Problems

In the method for producing a fructooligosaccharide-containing oil and fat according to the first aspect of the present invention, wherein the fructooligosaccharide is pulverized with at least the oil and fat and an emulsifier in a closed system pulverizer.

The fructooligosaccharide is preferably an amorphous substance.

The inventors of the present application have intensively studied “mass production of fructooligosaccharide-containing chocolate having low hygroscopicity”, and as a result, it was revealed that it can be realized by mixing fructooligosaccharide-containing oil and fat obtained by pulverizing fructooligosaccharide with at least oil and fat and emulsifier in a closed system pulverizer into a base of an oil-based confection or a raw material thereof, as described above. It is only speculative that this phenomenon is thought to be due to the fructooligosaccharide being coated with oil and fat.

The fructooligosaccharide-containing oil and fat according to the second aspect of the present invention contains fructooligosaccharide, fat, and emulsifier.

As a result of intensive studies by the inventors of the present application, it was revealed that the fructooligosaccharide-containing oil and fat can suppress moisture absorption of fructooligosaccharide without lowering the grinding efficiency at the time of grinding.

The fructooligosaccharide-containing oil and fat according to the third aspect of the present invention is the fructooligosaccharide-containing oil and fat according to the second aspect, and the fructooligosaccharide -containing oil and fat have a weight ratio of fructooligosaccharide to the oil and fat within a range of 0.5 or more and 2.5 or less, and a content of the emulsifier within a range of 0.1 wt % or more and 1.0 wt % or less .

As a result of intensive studies by the inventors of the present application, it was revealed that the fructooligosaccharide-containing oil and fat can further suppress moisture absorption of fructooligosaccharide without lowering the grinding efficiency at the time of grinding by setting the weight ratio of the fructooligosaccharide to the oil and fat within the above range and by setting the content of emulsifier within the above range in fructooligosaccharide-blended fats and oils.

The fructooligosaccharide-containing oil and fat according to the fourth aspect of the present invention is the fructooligosaccharide -containing oil and fat according to the second aspect or the third aspect, and the fructooligosaccharide-containing oil and fat maintain a moisture value of 5 wt % or less after 27 days from the time of production when stored at a temperature of 37° C. and a relative humidity of 50% after the production. The term “moisture value” used herein is synonymous with the terms “moisture content” and “moisture containing amount”.

The method for producing a fructooligosaccharide-containing oil-based confectionery according to the fifth aspect of the present invention includes a base preparation step and an oil-based confectionery producing step. In the base preparation step, the fructooligosaccharide-containing oil and fat obtained by the method for producing the fructooligosaccharide-containing oil and fat according to the first aspect or the fructooligosaccharide-containing oil and fat according to any one of the second through fourth aspects is mixed with the oil-based confectionery base or the raw material thereof to prepare a fructooligosaccharide-containing oil-based confectionery base. In the oil-based confectionery producing step, the fructooligosaccharide-containing oil-based confectionery base is used to produce a fructooligosaccharide-containing oil-based confectionery.

As a result of intensive studies on “mass production of fructooligosaccharide-containing oil-based confectionery with low hygroscopicity” by the inventors of the present application, it was revealed that this can be realized by mixing fructooligosaccharide-containing oil and fat into the oil-based confectionery base or the raw material thereof, as described above. It is only speculative that this phenomenon is thought to be due to the fructooligosaccharide being covered with fat and oil.

The fructooligosaccharide-containing oil-based confectionery according to the sixth aspect of the present invention maintains a moisture value of 3 wt % or less after 210 days from the time of production when stored in an unpackaged state at a temperature of 23° C. and a relative humidity of 38% after the production.

Therefore, the fructooligosaccharide -containing oil-based confectionery can satisfy low hygroscopicity over a long period of time.

The fructooligosaccharide-containing oil-based confection according to the seventh aspect of the present invention contains the fructooligosaccharide containing oil and fat obtained by the method for producing fructooligosaccharide-containing oil and fat according to the first aspect, or the fructooligosaccharide-containing oil and fat according to any one of the second aspect to the fourth aspect.

Therefore, the fructooligosaccharide -containing oil-based confectionery can realize low hygroscopicity.

The amorphous fructooligosaccharide-containing oil-based confectionery according to the eighth aspect of the present invention has a moisture value of 2.5 wt % or less.

The amorphous fructooligosaccharide-containing oil-based confectionery contains amorphous fructooligosaccharide.

Thus, the amorphous fructooligosaccharide-containing oil-based confectionery exhibits a good sweetness balance. The moisture value of the amorphous fructooligosaccharide -containing oil-based confectionery is 2.5 wt % or less as described above.

Therefore, the amorphous fructooligosaccharide-containing oil-based confectionery exhibits low hygroscopicity. Thus, the amorphous fructooligosaccharide-containing oil-based confectionery has good sweetness balance and low hygroscopicity.

### BRIEF EXPLANATION OF THE DRAWINGS

FIG. 1 is a graph showing the change over time of the moisture value in the fructooligosaccharide -containing paste according to working example 1 to 5 under an environment of a temperature of 37° C. and a relative humidity of 50%.

FIG. 2 is a particle size distribution of fructooligosaccharide -containing pastes according to working examples 3 to 5.

FIG. 3 is a graph showing the change over time of the moisture value in the fructooligosaccharide -containing paste according to working examples 6 to 9 under an environment of a temperature of 37° C. and a relative humidity of 50%.

FIG. 4 is a graph showing changes in viscosity over time in fructooligosaccharide -containing pastes according to working examples 1 to 5 under an environment of a temperature of 37° C. and a relative humidity of 50%.

FIG. 5 is a graph showing changes in viscosity over time in fructooligosaccharide -containing pastes according to working examples 6 to 9 under an environment of a temperature of 37° C. and a relative humidity of 50%.

FIG. 6 is a graph showing the time change of the moisture value in the fructooligosaccharide -containing milk chocolate according to working example 3 and the sucrose-containing milk chocolate according to reference example 1 under an environment of a temperature of 13° C. and a relative humidity of 38%.

FIG. 7 is a graph showing the time change of the moisture value in the fructooligosaccharide -containing milk chocolate according to working example 3 and the sucrose-containing milk chocolate according to reference example 1 in an environment of a temperature of 20° C. and a relative humidity of 38%.

FIG. 8 is a graph showing the time change of the moisture value in the fructooligosaccharide -containing milk chocolate according to working example 3 and the sucrose-containing milk chocolate according to reference example 1 under an environment of a temperature of 23° C. and a relative humidity of 38%.

FIG. 9 is a graph showing the change over time of the moisture value of fructooligosaccharide in an environment of a temperature of 20° C. and a relative humidity of 40%, and a temperature of 30° C. and a relative humidity of 40%.

### DESCRIPTION OF THE EMBODIMENTS

### —Method for Producing a Fructooligosaccharide-Containing Oil and Fat—

A method for producing a fructooligosaccharide-containing oil and fat according to an embodiment of the present invention includes a raw material preparation step and a pulverizing step. Hereinafter, these steps will be described in detail. These steps may be performed sequentially or continuously.

### (1) Raw Material Preparation Step

In the raw material preparation step, at least fructooligosaccharide, oil and fat (or sources of the oil and fat) and emulsifiers are prepared as raw materials for fructooligosaccharide-containing oil and fat. Here, as the oil and fat, an edible oil and fat may be used, or a cocoa ingredient such as cocoa mass, cocoa butter or the like may be used. The fructooligosaccharide is preferably blended within a range of 0.5 to 2.5 by weight relative to the oil and fat, more preferably within a range of 0.6 to 1.5 by weight. In another expression, the fructooligosaccharide : oil and fat (weight ratio) is preferably blended in the range of 35 to 70:65 to 30, more preferably in the range of 40 to 60:60 to 40. By setting the weight ratio within this range, moisture absorption of the fructooligosaccharide -containing oil and fat can be suppressed without lowering the grinding efficiency in the grinding step. Here, the moisture value in the oil and fat is preferably 0.2 wt % or less. The emulsifier is preferably within the range of 0.1 wt % or more and 1.0 wt % or less, more preferably within the range of 0.2 wt % or more and 0.5 wt % or less, based on the total amount of fructooligosaccharide-containing oil and fat.

### (2) Pulverizing Step

In the pulverizing step, the fructooligosaccharide is pulverized with at least the oil and fat, and the emulsifier in a closed pulverizer. As a result, a paste-like fructooligosaccharide -containing oil and fat is obtained.

Note that the moisture value in the paste-like fructooligosaccharide-containing oil and fat is preferably 5 wt % or less, more preferably 3 wt % or less, still more preferably 2 wt % or less, still more preferably 1 wt % or less, and particularly preferably 0.5 wt % or less, from the viewpoint of sufficiently reducing the moisture value of the fructooligosaccharide -containing oil-based confection (in the case where the fructooligosaccharide -containing oil-based confectionery is fructooligosaccharide -containing chocolate, from the viewpoint of making the moisture value of the fructooligosaccharide-containing chocolate 3 wt % or less as prescribed in the chocolate code).

Further, when this fructooligosaccharide -containing oil and fat is stored under an environment of a temperature of 37° C. and a relative humidity of 50% after production, it is preferable to maintain a moisture value of 5 wt % or less after 27 days from the time of production, more preferable to maintain a moisture value of 3 wt % or less, still more preferable to maintain a moisture value of 2 wt % or less, still more preferable to maintain a moisture value of 1 wt % or less, and particularly preferable to maintain a moisture value of 0.5 wt % or less.

Further, when the fructooligosaccharide -containing oil and fat is stored under the conditions of a temperature of 37° C. and a relative humidity of 50% after the time of production, it is preferable to maintain a viscosity of 70,000 cps or less after 27 days from the time of production, it is more preferable to maintain a viscosity of 50,000 cps or less, it is more preferable to maintain a viscosity of 30,000 cps or less, it is more preferable to maintain a viscosity of 10,000 cps or less, and it is particularly preferable to maintain a viscosity of 5,000 cps or less.

Further, when the fructooligosaccharide -containing oil and fat is stored under the conditions of a temperature of 37° C. and a relative humidity of 50% after the time of production, it is preferable to maintain a viscosity of 70,000 cps or less after 60 days from the time of production, it is more preferable to maintain a viscosity of 50,000 cps or less, it is more preferable to maintain a viscosity of 30,000 cps or less, it is more preferable to maintain a viscosity of 10,000 cps or less, and it is particularly preferable to maintain a viscosity of 5,000 cps or less.

Further, it is preferable that the fructooligosaccharide-containing oil and fat is stable in viscosity when stored under the conditions of “temperature 37° C. and relative humidity 50%” after production.

Incidentally, the closed system pulverizer is not particularly limited, but is, for example, a ball mill. It is preferable to use a wet pulverizer as the closed pulverizer. This is to avoid contact of the raw materials with humid air. In order to prevent excessive heat from being applied to the raw material at the time of raw material pulverization, it is preferable to appropriately adjust the input amount of the raw material, the flow rate, the number of revolutions and the like in accordance with the blending amount of the fructooligosaccharide, the type of the closed system pulverizer, and the like. This is because if the raw material is excessively heated at the time of pulverization, fructooligosaccharide is rubberized and burnt, or the flavor is changed.

—Method for producing a fructooligosaccharide-containing oil-based confectionery—

The method for producing a fructooligosaccharide-containing oil-based confectionery according to the embodiment of the present invention includes a raw material preparation step, a base preparation step, and an oil-based confectionery producing step. Hereinafter, these steps will be described in detail. These steps may be performed sequentially or continuously. Here, the oil-based confectionery is, for example, chocolate or the like. “Chocolate” as used herein is not limited to that by the provisions of the Fair Competition Codes concernig Labeling of Chocolates (National Chocolate Industry Fair Trade Council) or the provisions of laws and regulations, and refers to chocolate produced through a general chocolate producing step using a food oil and fat and a sugar as the main raw materials, and adding cocoa ingredients (cocoa mass, cocoa powder, cocoa butter, etc.), a dairy product, flavoring agent, an emulsifier, etc., as necessary. The chocolate includes plain chocolate, milk chocolate, white chocolate, dark chocolate, color chocolate, and the like.

### (1) Raw Material Preparation Step

In the raw material preparation step, at least fructooligosaccharide-containing oil and fat and oil-based confectionery base are prepared. Of course, in this step, raw materials other than the fructooligosaccharide-containing oil and fat and the oil-based confectionery base may be prepared. The fructooligosaccharide-containing oil and fat is purchased, or prepared according to the above-mentioned method for producing the fructooligosaccharide-containing oil and fat. The oil-based confectionery base may also be purchased or prepared according to conventional methods. When the oil-based confectionery base is a chocolate base, the raw materials include, for example, a food oil and fat, a sugar other than fructooligosaccharide, a cocoa ingredient (for example, cocoa mass, cocoa powder, cocoa butter, and the like), a dairy product (powdered milk and the like), a flavoring agent, an emulsifier, and the like.

### (2) Base Preparation Step

In the base preparation step, the fructooligosaccharide-containing oil and fat is mixed with the oil-based confectionery base or a raw material thereof to prepare fructooligosaccharide -containing oil-based confectionery base.

### (3) Oil-Based Confectionery Producing Step

In the oil-based confectionery producing step, the fructooligosaccharide -containing oil-based confectionery base is used to produce a fructooligosaccharide -containing oil-based confectionery. The producing referred to herein includes molding and the like.

The fructooligosaccharide-containing oil-based confectionery obtained as described above preferably maintains a moisture value of 3 wt % or less after 30 days from the time of production, more preferably maintains a moisture value of 3 wt % or less after 60 days from the time of production, more preferably maintains a moisture value of 3 wt % or less after 90 days from the time of production, more preferably maintains a moisture value of 3 wt % or less after 120 days from the time of production, more preferably maintains a moisture value of 3 wt % or less after 150 days from the time of production, more preferably maintains a moisture value of 3 wt % or less after 180 days from the time of production, and particularly preferably maintains a moisture value of 3 wt % or less after 210 days from the time of production when the fructooligosaccharide -containing oil-based confectionery is stored in an unpackaged state under the conditions of a temperature of 23° C. and a relative humidity of 38% after the time of production,

### <Features of Fructooligosaccharide-Containing Oil and Fat and Methods for Producing the Same According to the Present Embodiment>

(1)

The fructooligosaccharide-containing oil and fat according to the present embodiment can sufficiently suppress moisture absorption of the fructooligosaccharide. Therefore, the fructooligosaccharide -containing oil and fat can contribute to mass production of a fructooligosaccharide-containing oil-based confectionery having low hygroscopicity.

(2)

In the refiner, which is a conventional milling method, the surface area of the powder containing the fructooligosaccharide is increased, and the hygroscopicity of the fructooligosaccharide is increased. Therefore, when this refiner is used, it becomes difficult to observe the moisture value in the oil-based confectionery and the fructooligosaccharide content. On the other hand, in the method for producing a fructooligosaccharide-containing oil and fat according to the present embodiment, the fructooligosaccharide is pulverized together with at least the oil and fat, and the emulsifier in a closed system pulverizer. For this reason, in this method for producing fructooligosaccharide-containing oil and fat, it is possible to easily adjust the blending amount of fructooligosaccharide into an oil-based confection and to sufficiently suppress moisture absorption of fructooligosaccharide.

### <Features of Fructooligosaccharide-Containing Oil-Based Confectionery According to the Present Embodiment>

(1)

The fructooligosaccharide-containing oil-based confectionery according to the present embodiment has low hygroscopicity. Therefore, the fructooligosaccharide -containing oil-based confectionery can be stored for a long time in a good environment. When the fructooligosaccharide-containing oil-based confectionery is fructooligosaccharide -containing chocolate, the fructooligosaccharide-containing chocolate can maintain the moisture value prescribed in the chocolate code for a long period of time. The fructooligosaccharide -containing oil-based confectionery is less rough and can give a good texture to the consumers.

(2)

In the fructooligosaccharide-containing oil-based confectionery according to the present embodiment, when amorphous fructooligosaccharide is selected as the fructooligosaccharide, the sweetness balance can be made better than when kestose is selected.

### <Working Examples and Reference Examples>

Hereinafter, the present invention will be described in more detail by showing working examples and reference examples. The present invention is not limited to the following examples.

### WORKING EXAMPLE 1

### 1. Preparation of Fructooligosaccharide-Containing Paste

A fructooligosaccharide-containing paste was prepared by mixing 69.8 parts by weight of amorphous fructooligosaccharide (Meioligo P from Meiji Food Material), 29.9 parts by weight of cocoa butter (moisture value: 0.16-0.17 wt %) and 0.3 parts by weight of lecithin with a mixer (see Table 1) and milling the resulting mixture with a ball mill for 60 minutes to obtain a fructooligosaccharide-containing paste. The oil content in the fructooligosaccharide-containing paste was 30.2 wt %, see Table 2. The oil content in the fructooligosaccharide-containing pastes was measured according to “Analytical Methods of Annex Nutritional Components, etc” 2. Lipids (4) Acid Decomposition Method in.“Annex Nutritional Labeling”(http://www.caa.go.jp/policies/policy/food_labeling/food_labeling_act/pdf/foods_index_18_180119_0003.pdf))) on the website of the Consumer Affairs Agency of Japan. As a reference, FIG. 9 shows the change over time of the moisture value of the fructooligosaccharide in an environment of a temperature of 20° C. and a relative humidity of 40% and a temperature of 30° C. and a relative humidity of 40%.

### 2. Preparation of Fructooligosaccharide-Containing Milk Chocolate

The fructooligosaccharide-containing paste obtained as described above was mixed with milk chocolate base (produced by Meiji Corporation) so that the fructooligosaccharide occupied 14.5 wt % in a target fructooligosaccharide-containing milk chocolate, and then the milk chocolate base was molded according to the usual method to obtain the target fructooligosaccharide-containing milk chocolate.

### 3. Rating of Physical Properties of Mixtures and Fructooligosaccharide-Containing Pastes

### (1) Rating of the Grindability of the Mixture

The 50% particle diameter (median diameter) D50 of the pulverized product obtained by pulverizing the above mixture for 1 hour was measured, and this was rated in five grades of “1” to “5”. Note that “1” was assigned to those with a D50 value of 50 μm or more, “2” was assigned to those with a D50 value within the range of 30 μm or more and less than 50 μm, “3” was assigned to those with a D50 value within the range of 13 μm or more and less than 30 μm, “4” was assigned to those with a D50 value within the range of 8 μm or more and less than 13 μm, and “5” was assigned to those with a D50 value of less than 8 μm. That is, the closer to “1”, the lower the grindability is, and the closer to “5”, the higher the grindability is. The rating of the mixture according to this working example was “4” (see Table 3). Incidentally, the 50% particle diameter (median diameter) D50 of the pulverized product was measured using a laser analysis type particle size distribution measuring instrument SALD-2200 manufactured by Shimadzu Corporation.

### (2) Rating of Hygroscopicity of Fructooligosaccharide-Containing Paste

The fructooligosaccharide-containing paste obtained as described above was stored in an environment of “temperature 37° C. and relative humidity 50%” over 27 days, and the moisture value in the fructooligosaccharide-containing paste was measured at the start of storage, at the elapse of 1 day, at the elapse of 3 days, at the elapse of 14 days, and at the elapse of 27 days. The moisture value was measured according to the “Analytical Methods for Annexed Nutritional Ingredients, etc” 5. Carbohydrate I moisture (4) Atmospheric oven-drying method in.“Annexed Nutritional Labeling”(http://www.caa.go.jp/policies/policy/food_labeling/food_labeling_act/pdf/foods_index_18_180119_0003.pdf) on the website of the Consumer Affairs Agency of Japan).

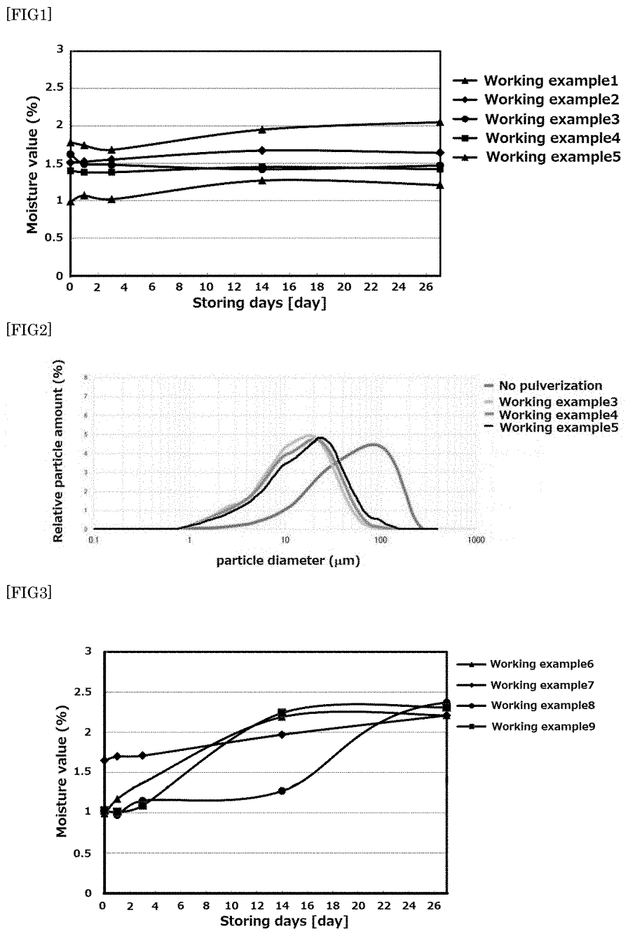

The results in an environment of a temperature of 37° C. and a relative humidity of 50% are shown in FIG. 1. As shown in FIG. 1, the fructooligosaccharide-containing paste according to this working example exhibited sufficient low hygroscopicity over 27 days in the environment of “temperature 37° C. and relative humidity 50%”. Here, sufficient low hygroscopicity means that the moisture value in the fructooligosaccharide-containing paste is 5 wt % or less. Then, a value (i.e., the moisture change amount) obtained by subtracting the “moisture value in the fructooligosaccharide-containing paste at the time of storage start (initial)” from the “moisture value in the fructooligosaccharide-containing paste at the time of 27 days” was divided by the fructooligosaccharide content in the fructooligosaccharide-containing paste (this value is hereinafter referred to as the “moisture change rate”) and rated in five grades of “1” to “5”. Here, “1” is assigned to those with a moisture change rate of 6% or more, “2” is assigned to those with a moisture change rate of 1% or more and less than 6%, “3” is assigned to those with a moisture change rate of 0.8% or more and less than 1%, “4” is assigned to those with a moisture change rate of 0.2% or more and less than 0.8%, and “5” is assigned to those with a moisture change rate of less than 0.2%. That is, the closer to “1”, the higher the hygroscopicity is, and the closer to “5”, the lower the hygroscopicity is. The rating of the fructooligosaccharide-containing paste according to this working example was “4” (see Table 3).

(3) Change in Viscosity of Fructooligosaccharide-Containing Paste

The fructooligosaccharide-containing paste obtained as described above was stored in an environment of “temperature 37° C. and relative humidity 50%” for 60 days, and the viscosity of the fructooligosaccharide-containing paste was measured at the start of storage, at the elapse of 1 day, at the elapse of 3 days, at the elapse of 14 days, at the elapse of 27 days, and at the elapse of 60 days. The viscosity was measured using a Type B viscometer. At this time, a rotor No. 6 was used as the rotor, and the rotation speed thereof was set to 4rpm. The measured temperature was the storage temperature zone of each paste.

The results are shown in FIG. 4. As shown in FIG. 4, the viscosity of this fructooligosaccharide-containing paste was 60,000 cps or less and its behavior was stable until the elapse of 27 days, but increased to nearly 70,000 cps at the elapse of 60 days.

### (4) Sensory Rating of Texture

The fructooligosaccharide-containing milk chocolate obtained as described above was eaten by 5 trained specialist panels, and the texture was rated in 5 grades of “1” to “5”. The results of the rating were determined by consultation of five expert panels. It should be noted that “1” is assigned to those that feel strongly rough in the mouth, “2” is assigned to those that feel rough in the mouth, “3” is assigned to those that feel slightly rough in the mouth, “4” is assigned to those that feel slightly smooth in the mouth, and “5” is assigned to those that feel smooth in the mouth. That is, the closer to “1”, the greater the sense of roughness, and the closer to “5”, the greater the sense of smoothness. The rating of the fructooligosaccharide-containing milk chocolate according to this working example was “5” (see Table 3).

### (5) Overall Rating

The ratings of (1), (2) and (4) described above were rated in three-level of “A”, “B” and “C”. “A” means that all the ratings of (1), (2) and (4) were highly rated, “B” means that a slightly inferior rating is included “C” means that a relatively inferior rating is included. However, in any of the above overall rating, there is no problem in selling the fructooligosaccharide-containing milk chocolate as a commercial product. The overall rating of the fructooligosaccharide-containing milk chocolate according to this working example was “B”.

### WORKING EXAMPLE 2

A fructooligosaccharide-containing paste and fructooligosaccharide-containing milk chocolate were prepared in the same manner as in the working example 1 except that the additive amount of amorphous fructooligosaccharide was 59.8 parts by weight and the additive amount of cocoa butter was 39.9 parts by weight (see Table 1), and were rated in the same manner as in the working example 1. The oil content in the fructooligosaccharide-containing paste was 40.2 wt % (see Table 2).

The rating results were as shown in Table 3, FIG. 1 and FIG. 4. As shown in FIG. 1, the fructooligosaccharide-containing paste according to this working example exhibited sufficient low hygroscopicity over 27 days in the environment of “temperature 37° C. and relative humidity 50%”. The rating of the hygroscopicity of this fructooligosaccharide-containing paste was “5”. The grindability and texture were rated to be equivalent to the grindability and texture of the fructooligosaccharide-containing paste according to the working example 1. For this reason, the overall rating was “A”. As shown in FIG. 4, the viscosity of the fructooligosaccharide-containing paste was 20,000 cps or less and the behavior thereof was stable until the elapse of 27 days but increased to slightly higher than 20,000 cps at the elapse of 60 days.

### WORKING EXAMPLE 3

A fructooligosaccharide-containing paste and fructooligosaccharide-containing milk chocolate were prepared in the same manner as in the working example 1 except that the additive amount of amorphous fructooligosaccharide was 49.9 parts by weight and the additive amount of cocoa butter was 49.8 parts by weight (see Table 1), and were rated in the same manner as in the working example 1. The oil content in the fructooligosaccharide-containing paste was 50.1 wt % (see Table 2).

The rating results were as shown in Table 3, FIG. 1 and FIG. 4. As shown in FIG. 1, the fructooligosaccharide-containing paste according to this working example exhibited sufficient low hygroscopicity over 27 days in the environment of “temperature 37° C. and relative humidity 50%”. The rating of the hygroscopicity of this fructooligosaccharide-containing paste was “5”. The grindability and texture were rated to be equivalent to the grindability and texture of the fructooligosaccharide-containing paste according to the working example 1. For this reason, the overall rating was “A”. As shown in FIG. 4, the viscosity of the fructooligosaccharide-containing paste was 10,000 cps or less and the behavior thereof was stable until 60 days elapsed.

In addition, in this working example, after the above-mentioned mixtures were pulverized, the particle size distribution of the pulverized product was measured using a laser analysis type particle size distribution measuring instrument SALD-2200 manufactured by Shimadzu Corporation. The measurement results are shown in FIG. 2. The pulverized product had a 50% diameter (median diameter) D50 of 9.80 μm and a 95% particle diameter D95 of 33.1 μm.

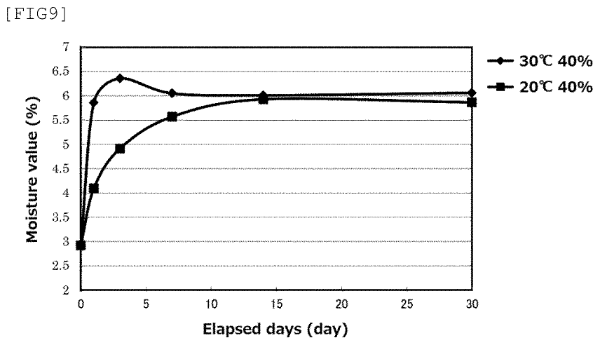

Further, the fructooligosaccharide-containing milk chocolate obtained as described above was stored for 210 days in the respective environments of “temperature 13° C. and relative humidity ”%, “temperature 20° C. and relative humidity 38%” and “temperature 23° C. and relative humidity 38%”, and the moisture values in the fructooligosaccharide-containing milk chocolate at the start of storage, at the elapse of 1 day, at the elapse of 7 days, at the elapse of 30 days, at the elapse of 90 days and at the elapse of 210 days were measured. The results are shown in FIGS. 6, 7 and 8. The moisture value was measured according to the atmospheric pressure oven-drying method described in the working example 1. The fructooligosaccharide-containing milk chocolate according to this working example exhibited good low hygroscopicity over 210 days in any environment. Incidentally, when the time change of the hygroscopicity of the fructooligosaccharide-containing milk chocolate in the relatively severe humidity environment of “temperature 20° C. and relative humidity 70%” and “temperature 23° C. and relative humidity 70%” was measured by the same method as described above, the fructooligosaccharide-containing milk chocolate was able to maintain the moisture value of 3 wt % or less for 7 days.

### WORKING EXAMPLE 4

A fructooligosaccharide-containing paste and fructooligosaccharide-containing milk chocolate were prepared in the same manner as in the working example 1 except that the additive amount of amorphous fructooligosaccharide was 39.9 parts by weight and the additive amount of cocoa butter was 59.8 parts by weight (see Table 1), and were rated in the same manner as in the working example 1. The oil content in the fructooligosaccharide-containing paste was 60.1 wt %, (see Table 2).

The rating results were as shown in Table 3, FIG. 1 and FIG. 4. As shown in FIG. 1, the fructooligosaccharide-containing paste according to this working example had a very low moisture value at the initial stage (i.e., at the start of storage), maintained its moisture value over 27 days in an environment of “temperature 37° C. and relative humidity 50%”, and exhibited a very low hygroscopicity. The rating of the hygroscopicity of this fructooligosaccharide-containing paste was “5”. The grindability and texture were rated to be equivalent to the grindability and texture of the fructooligosaccharide-containing paste according to the working example 1. For this reason, the overall rating was “A”. As shown in FIG. 4, the viscosity of the fructooligosaccharide-containing paste was 5,000 cps or less and the behavior thereof was stable until 60 days elapsed.

In addition, in this working example, after the above-mentioned mixtures were pulverized, the particle size distribution of the pulverized product was measured using a laser analysis type particle size distribution measuring instrument SALD-2200 manufactured by Shimadzu Corporation. The measurement results are shown in FIG. 2. The pulverized product had a 50% diameter (median diameter) D50 of 12.6 μm and a 95% particle diameter D95 of 44.9 μm.

### WORKING EXAMPLE 5

A fructooligosaccharide-containing paste and fructooligosaccharide-containing milk chocolate were prepared in the same manner as in the working example 1 except that the additive amount of amorphous fructooligosaccharide was 33.2 parts by weight and the additive amount of cocoa butter was 66.5 parts by weight (see Table 1), and were rated in the same manner as in the working example 1.The oil content in the fructooligosaccharide-containing paste was 66.8 wt % (see Table 2).

The rating results were as shown in Table 3, FIG. 1 and FIG. 4. As shown in FIG. 1, the fructooligosaccharide-containing paste according to this working example had a very low moisture value at the initial stage (i.e., at the start of storage), maintained its moisture value over 27 days in an environment of “temperature 37° C. and relative humidity 50%”, and exhibited a very low hygroscopicity. The rating of the hygroscopicity of this fructooligosaccharide-containing paste was “4”. The rating of the grindability and texture were both “3”, which was inferior to the rating of the grindability and texture of the fructooligosaccharide-containing paste according to the working example 1. For this reason, the overall rating was “B”. As shown in FIG. 4, the viscosity of the fructooligosaccharide-containing paste was 5,000 cps or less and the behavior thereof was stable until 60 days elapsed.

In addition, in this working example, after the above-mentioned mixtures were pulverized, the particle size distribution of the pulverized product was measured using a laser analysis type particle size distribution measuring instrument SALD-2200 manufactured by Shimadzu Corporation. The measurement results are shown in FIG. 2. The pulverized product had a 50% diameter (median diameter) D50 of 14.9 μm and a 95% particle diameter D95 of 56.4 μm.

### WORKING EXAMPLE 6

A fructooligosaccharide-containing paste and a fructooligosaccharide-containing milk chocolate were prepared in the same manner as in the working example 1 except that the additive amount of amorphous fructooligosaccharide was 35.6 parts by weight and 29.9 parts by weight of cocoa butter was replaced with 64.1 parts by weight of cocoa mass (see Table 1), and were rated in the same manner as in the working example 1. The oil content in the fructooligosaccharide-containing paste was 36.1 wt % (see Table 2).

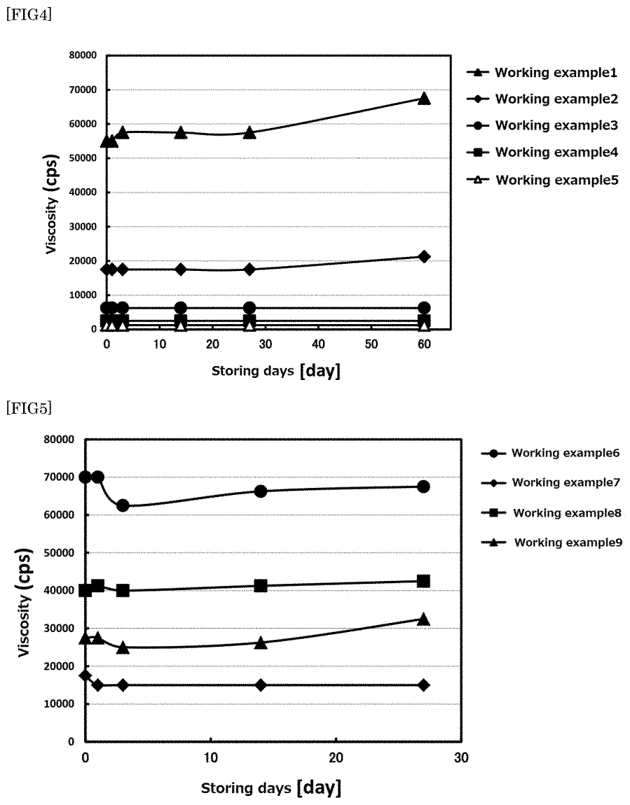

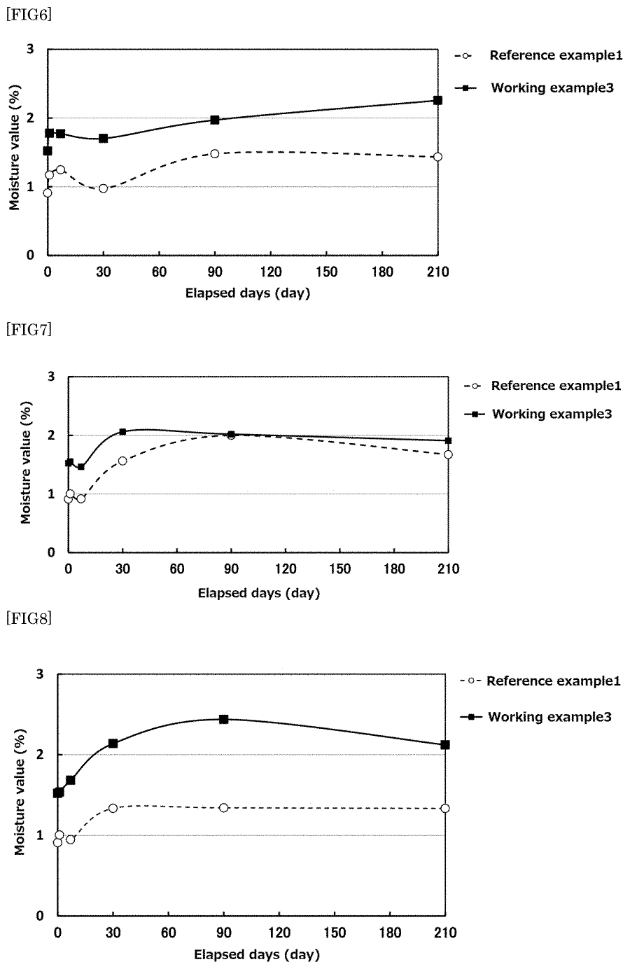

The measurement and rating results are shown in Table 3, FIG. 3 and FIG. 5. As shown in FIG. 3, the fructooligosaccharide-containing paste according to this working example exhibited a sufficiently low hygroscopicity over 27 days, although an increasing tendency of the moisture value was observed in the environment of “temperature 37° C. and relative humidity 50%”. The rating of the hygroscopicity of this fructooligosaccharide-containing paste was “2”. The grindability was equivalent to the grindability of the fructooligosaccharide-containing paste according to the working example 1, but the rating of the texture was “4”, which was slightly inferior to the rating of the texture of the fructooligosaccharide-containing paste according to the working example 1. For this reason, the overall rating was “C”. As shown in FIG. 5, the viscosity of the fructooligosaccharide-containing paste was 70,000 cps or less and the behavior thereof was stable until the elapse of 27 days.

### WORKING EXAMPLE 7

A fructooligosaccharide-containing paste and a fructooligosaccharide-containing milk chocolate were prepared in the same manner as in the working example 1 except that the additive amount of amorphous fructooligosaccharide was 49.9 parts by weight, the additive amount of cocoa butter was 24.9 parts by weight, and further 24.9 parts by weight of cocoa mass was added (see Table 1), and were rated in the same manner as in the working example 1. The oil content in the fructooligosaccharide-containing paste was 39.2 wt % (see Table 2).

The measurement and rating results are shown in Table 3, FIG. 3 and FIG. 5. As shown in FIG. 3, the fructooligosaccharide-containing paste according to this working example exhibited a sufficiently low hygroscopicity over 27 days, although an increasing tendency of the moisture value was observed in the environment of “temperature 37° C. and relative humidity 50%”. The rating of the hygroscopicity of this fructooligosaccharide-containing paste was “2”. The grindability was equivalent to the grindability of the fructooligosaccharide-containing paste according to the working example 1, but the rating of the texture was “4”, which was slightly inferior to the rating of the texture of the fructooligosaccharide-containing paste according to the working example 1. For this reason, the overall rating was “C”. As shown in FIG. 5, the viscosity of the fructooligosaccharide-containing paste was 20,000 cps or less and the behavior thereof was stable until the elapse of 27 days.

### WORKING EXAMPLE 8

A fructooligosaccharide-containing paste and a fructooligosaccharide-containing milk chocolate were prepared in the same manner as in the working example 1 except that the additive amount of amorphous fructooligosaccharide was 27.0 parts by weight and 29.9 parts by weight of cocoa butter was replaced with 72.7 parts by weight of cocoa mass (see Table 1), and were rated in the same manner as in the working example 1. The oil content in the fructooligosaccharide-containing paste was 40.9 wt % (see Table 2).

The measurement and rating results are shown in Table 3, FIG. 3 and FIG. 5. As shown in FIG. 3, the fructooligosaccharide-containing paste according to this working example exhibited a sufficiently low hygroscopicity over 27 days, although an increasing tendency of the moisture value was observed in the environment of “temperature 37° C. and relative humidity 50%”. The rating of the hygroscopicity of this fructooligosaccharide-containing paste was “2”. The grindability was equivalent to the grindability of the fructooligosaccharide-containing paste according to the working example 1, but the rating of the texture was “4”, which was slightly inferior to the rating of the texture of the fructooligosaccharide-containing paste according to the working example 1. For this reason, the overall rating was “C”. As shown in FIG. 5, the viscosity of the fructooligosaccharide-containing paste stably remained in the vicinity of 40,000 cps until the elapse of 27 days.

### WORKING EXAMPLE 9

A fructooligosaccharide-containing paste and a fructooligosaccharide-containing milk chocolate were prepared in the same manner as in the working example 1 except that the additive amount of amorphous fructooligosaccharide was 21.7 parts by weight and 29.9 parts by weight of cocoa butter was replaced with 78.0 parts by weight of cocoa mass (see Table 1), and were rated in the same manner as in the working example 1. The oil content in the fructooligosaccharide-containing paste was 43.9 wt % (see Table 2).

The measurement and rating results are shown in Table 3, FIG. 3 and FIG. 5. As shown in FIG. 3, the fructooligosaccharide-containing paste according to this working example exhibited a sufficiently low hygroscopicity over 27 days, although an increasing tendency of the moisture value was observed in the environment of “temperature 37° C. and relative humidity 50%”. The rating of the hygroscopicity of this fructooligosaccharide-containing paste was “2”. The grindability was equivalent to the grindability of the fructooligosaccharide-containing paste according to the working example 1, but the rating of the texture was “4”, which was slightly inferior to the rating of the texture of the fructooligosaccharide-containing paste according to the working example 1. For this reason, the overall rating was “C”. As shown in FIG. 5, the viscosity of the fructooligosaccharide-containing paste was 30,000 cps or less and its behavior was stable until the elapse of 14 days, but increased to slightly over 30,000 cps at the elapse of 27 days.

TABLE 1

| | Working | Working | Working | Working | Working | Working | Working | Working | Working |
|---|---|---|---|---|---|---|---|---|---|
| | example 1 | example 2 | example 3 | example 4 | example 5 | example 6 | example 7 | example 8 | example 9 |
| Fructooligosaccharide | 69.8 | 59.8 | 49.9 | 39.9 | 33.2 | 35.6 | 49.9 | 27.0 | 21.7 |
| (wt %) | | | | | | | | | |
| Cocoa mass (wt %) | 0 | 0 | 0 | 0 | 0 | 64.1 | 24.9 | 72.7 | 78.0 |
| Cocoa butter (wt %) | 29.9 | 39.9 | 49.8 | 59.8 | 66.5 | 0 | 24.9 | 0 | 0 |
| Lecithin (wt %) | 0.3 | 0.3 | 0.3 | 0.3 | 0.3 | 0.3 | 0.3 | 0.3 | 0.3 |
| Total (wt %) | 100 | 100 | 100 | 100 | 100 | 100 | 100 | 100 | 100 |

TABLE 2

| | Working | Working | Working | Working | Working | Working | Working | Working | Working |
|---|---|---|---|---|---|---|---|---|---|
| | example 1 | example 2 | example 3 | example 4 | example 5 | example 6 | example 7 | example 8 | example 9 |
| Oil content (wt %) | 30.2 | 40.2 | 50.1 | 60.1 | 66.8 | 36.1 | 39.2 | 40.9 | 43.9 |

TABLE 3

| | Working | Working | Working | Working | Working | Working | Working | Working | Working |
|---|---|---|---|---|---|---|---|---|---|
| | example 1 | example 2 | example 3 | example 4 | example 5 | example 6 | example 7 | example 8 | example 9 |
| Hygroscopicity | 4 | 5 | 5 | 5 | 4 | 2 | 2 | 2 | 2 |
| Grindability | 4 | 4 | 4 | 4 | 3 | 4 | 4 | 4 | 4 |
| Sensory rating | 5 | 5 | 5 | 5 | 3 | 4 | 4 | 4 | 4 |
| Overall rating | B | A | A | A | B | C | C | C | C |

TABLE 4

| | Working | Working | Working |
|---|---|---|---|
| | example 3 | example 4 | example 5 |
| D50 (μm) | 9.80 | 12.6 | 14.9 |
| D95 (μm) | 33.1 | 44.9 | 56.4 |
| Sensory (roughness) | A | A | B |

### Reference Example 1

Ordinary milk chocolate was prepared from the raw material described in Example 3 instead of sucrose (the compounding ratio was the same) by a conventional method (including a refining step using a refiner and a conching step).

The ordinary milk chocolate obtained as described above was stored for 210 days in the respective environments of “temperature 13° C. and relative humidity 38%”, “temperature 20° C. and relative humidity 38%” and “temperature 23° C. and relative humidity 38%”, and the moisture values in the ordinary milk chocolate were measured at the start of storage, at the elapse of 1 day, at the elapse of 7 days, at the elapse of 30 days, at the elapse of 90 days and at the elapse of 210 days. The results are shown in FIGS. 6, 7 and 8. The moisture value was measured according to the atmospheric pressure oven-drying method described in the working example 1. The ordinary milk chocolate according to this reference example exhibited good low hygroscopicity over 210 days in any environment.

### Reference Example 2

Referring to the working example 1 of Japanese Unexamined Patent Application Publication No. 9-65829, a crystalline kestose-containing chocolate was produced.

### Verification Example 1

The fructooligosaccharide-containing milk chocolate obtained in the working example 3 and the crystalline kestose-containing chocolate of the reference Example 2 were eaten by five trained specialist panels to select a sweeter feeler. As a result, all five expert panels selected the fructooligosaccharide-containing milk chocolate of the working example 3. The opinion of the expert panel included the comment that the fructooligosaccharide-containing milk chocolate of the working example 3 had a better sweetness balance than the crystalline kestose-containing chocolate of the reference example 2.

### SUMMARY

(1)

The fructooligosaccharide-containing paste according to the working examples 1 to 9 all exhibited extremely good low hygroscopicity in an environment of temperature 37° C. and relative humidity 50% with a moisture value of less than 5 wt % over 27 days (see FIGS. 1 and 3).

(2)

From the results shown in FIG. 1 and FIG. 3, it was found that the hygroscopicity of the fructooligosaccharide-containing paste became lower as the amount of oil in the paste increased.

(3)

The hygroscopicity of the fructooligosaccharide-containing pastes of the working examples 1 to 5 using cocoa butter as the oil source was more stable than the hygroscopicity of the fructooligosaccharide-containing pastes of the working examples 6 to 9 using cocoa mass as the oil source. It is only inferred that this is probably because the fructooligosaccharide-containing paste according to the working examples 6 to 9 was affected by moisture derived from cocoa mass. Therefore, when low hygroscopicity is required for the fructooligosaccharide-containing oil-based confectionery, cocoa butter is more preferable than cocoa mass as the oil source.

(4)

Under the relatively mild environment of “temperature 13° C. and relative humidity 38%”, “temperature 20° C. and relative humidity 38%”, and “temperature 23° C. and relative humidity 38%”, it was found that the fructooligosaccharide-containing milk chocolate, as is the case in ordinary milk chocolate, has a moisture value of less than 3 wt % over 210 days, exhibits very good low hygroscopicity, and can satisfy the moisture value definition specified in the chocolate code for a long period of time.

(5)

In the production of the fructooligosaccharide-containing paste, it was revealed that the texture of the fructooligosaccharide-containing chocolate prepared from the obtained fructooligosaccharide-containing paste was smoothed by pulverizing the raw material until the median diameter D50 was 13 μm or less and the 95% particle diameter D95 was 45 μm or less.

(6)

In the production of the fructooligosaccharide-containing paste, it has been revealed that the pulverization suitability does not greatly depend on the type and the blending amount of the oil and fat source.

(7)

It was revealed that the fructooligosaccharide-containing paste exhibited a relatively stable viscosity behavior in the environment of “temperature 37° C. and relative humidity 50%”.

### INDUSTRIAL APPLICABILITY

The method for producing fructooligosaccharide-containing oil-based confectionery according to the present invention can be used not only for fructooligosaccharide-containing chocolate but also for mass production of other oil-based confectionery.

1. A method for producing a fructooligosaccharide-containing oil and fat, wherein the fructooligosaccharide is pulverized with at least the oil and fat, and an emulsifier in a closed system pulverizer.

2. A fructooligosaccharide-containing oil and fat produced by the method for producing the fructooligosaccharide-containing oil and fat according to claim 1.

3. A fructooligosaccharide-containing oil and fat comprising:
a fructooligosaccharide,
an oil and fat, and
an emulsifier.

4. The fructooligosaccharide-containing oil and fat according to claim 3,
wherein the weight ratio of the fructooligosaccharide to the oil and fat is within the range of 0.5 to 2.5,
the content of the emulsifier is within the range of 0.1 wt % or more and 1.0 wt % or less.

5. The fructooligosaccharide containing oil and fat according to claim 3,
wherein the moisture value of 5 wt % or less is maintained after 27 days from the time of production when stored at a temperature of 37° C. and a relative humidity of 50% after the production.

6. A method for producing a fructooligosaccharide-containing oil-based confectionery comprising:
a base preparation step for preparing a fructooligosaccharide-containing oil-based confectionary base by mixing the fructooligosaccharide-containing oil and fat according to claim 2 with an oil-based confectionary base or a raw material thereof, and
an oil-based confectionery producing step for producing a fructooligosaccharide-containing oil-based confectionery using the fructooligosaccharide-containing oil-based confectionery base.

7. A fructooligosaccharide-containing oil-based confectionery produced by the method for producing a fructooligosaccharide-containing oil-based confectionery according to claim 6.

8. A fructooligosaccharide-containing oil-based confectionery comprising:
wherein the moisture value of 3 wt % or less is maintained after 210 days from the time of production when stored in an unpackaged state at a temperature of 23° C. and a relative humidity of 38% after the production.

9. An amorphous fructooligosaccharide-containing oil-based confection having a moisture value of 2.5 wt % or less.

10. The fructooligosaccharide containing oil and fat according to claim 4,
wherein the moisture value of 5 wt % or less is maintained after 27 days from the time of production when stored at a temperature of 37° C. and a relative humidity of 50% after the production.
